(19) United States
(12) Patent Application Publication
(10) Pub. No.: US 2010/0021936 A1
(43) Pub. Date: Jan. 28, 2010

(54) Sensor for Spores

(76) Inventors: Graham Christie, Cambridge (GB); Christopher Robin Lowe, Cambridge (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/522,462

(22) PCT Filed: Jan. 17, 2008
(86) PCT No.: PCT/GB08/00145

§ 371 Date: Oct. 6, 2009

(30) Foreign Application Priority Data
Jan. 17, 2007 (GB) 0700885.7

(51) Int. Cl. G01N 33/53; C12M 1/00

(52) U.S. Cl. 435/7.1; 435/287.2

(57) ABSTRACT

A sensor for spores, comprises spore-binding ligands and, on or within the body of the sensor, a material that is responsive to Ca-DPA (calcium-dipicolinic acid) but not to a (or another) germinant.

### FIELD OF THE INVENTION

This invention relates to a pathogen sensor, and in particular to a sensor for spores.

### BACKGROUND OF THE INVENTION

The rapid detection and identification of pathogenic microorganisms is increasingly important in a number of clinical, environmental and bio-defence applications. However, the definitive identification of a microbial pathogen remains a time-consuming laboratory based procedure, despite the fact that a number of technologies are available, such as ELISA and PCR, to aid this process. Unfortunately, both ELISA and PCR-based techniques are associated with a number of disadvantages, such as poor sensitivity and/or complex sample preparation and the requirement for highly trained personnel. A particular need is to develop a rapid diagnostic platform, primarily for the detection of Bacillus anthracis (the causative agent for anthrax), but which also has the potential to be extended to detect other organisms that may be of interest.

It would be desirable to detect germination per se. One of the earliest events associated with germination is the release of the spore's depot of DPA, i.e. dipicolinic acid, which forms a chelate with endogenous divalent metal ions (predominantly calcium).

Holographic sensors are known. Reference may be made, for example, to WO95/26499, WO99/63408 and other publications in the name of Smart Holograms Limited.

### SUMMARY OF THE INVENTION

The present invention is based on a system where the release of Ca-DPA from captured target spores is utilised to activate components of the germination apparatus, typically hydrolytic enzymes, in proximity to an appropriate holographic or other sensor. Essentially this design transduces and amplifies the target spore germination response, to increase the sensitivity of the sensor.

According to first aspect of the present invention, a sensor for spores comprising spore-binding ligands and, on or within the body of the sensor, a material that is responsive to Ca-DPA but not to a (or another) germinant.

According to a second aspect of the present invention, a method for the detection of spores in a sample, comprises:

contacting the sample with a sensor as defined above, whereby spores in the sample are bound by the ligands;

introducing the germinant; and

observing the response of the material.

### BRIEF DESCRIPTION OF THE DRAWINGS

FIG. 1 is a schematic representation of a Ca-DPA activated/enzyme-linked holographic sensor.

FIG. 2 is a graph showing the effect of Ca²⁺-DPA-activated GPR^{S} on the diffraction intensity of an SASP-based hologram, after activated GPR^{S} was added to a SSAP hologram equilibrated in 25 mM Tris-HCl, 5 mM CaCl₂, pH 7.4 at 37° C. to a final concentration of 50 μg/ml.

FIG. 3 is a graph of B. megaterium QM B1551 “receptorless” spore germination response.

### DESCRIPTION OF PREFERRED EMBODIMENTS

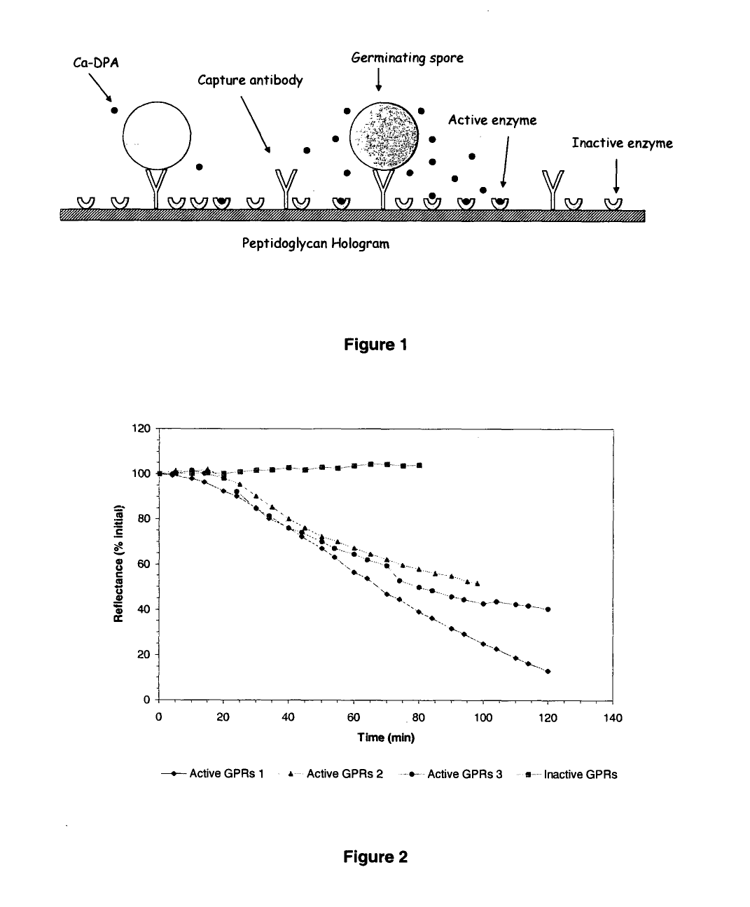

The invention will be described by way of example only with reference to a schematic representation of a sensor embodying the invention, illustrated in FIG. 1.

To demonstrate the principle of this sensor design, a holographic sensor was made, comprising small acid-soluble proteins (SASP's) extracted from dormant Bacillus megaterium KM spores. These small peptides protect the spore DPA during dormancy and are rapidly degraded during germination, to provide a source of amino acids for de novo protein synthesis. The enzyme responsible for SASP hydrolysis, germination protease (GPR), is known to be activated by Ca-DPA. Thus, recombinant germination protease (GPR) was prepared and incubated with a SASP hologram. FIG. 2 demonstrates that, upon addition of Ca-DPA (50 mM), the enzyme is activated and degrades the hologram, while holograms incubated in the presence of GPR minus Ca-DPA remain stable.

One approach to the invention is to employ recombinant CwlJ, a cortex lytic enzyme activated by Ca-DPA, and to utilize the activity of this enzyme on peptidoglycan-containing holograms are continuing. In an alternative approach, e.g. in case there may be problems associated with stability/activity of purified recombinant enzymes, intact spores (as opposed to recombinant components) may be used as amplification vectors. An example is the use of genetically modified “receptorless” Bacillus megaterium spores that can only germinate in response to exogenous Ca-DPA via activation of the spore-coat located cortex-lytic enzyme CwlJ. These spores germinate in response to Ca-DPA, e.g. at a concentration in the region of ˜30 mM. The structural gene for Bacillus megaterium CwlJ may be cloned, in order to prepare a construct that over-expresses the gene, to increase the sensitivity of this system. Such vector amplification spores can be integrated with RNTA divalent metal ion-sensitive polymers, to provide a means of built-in signal amplification.

It has been found that antibody-immobilised spores retain the ability to germinate. Essentially, purified polyclonal antisera raised against de-activated spore suspensions were immobilised onto amino-silanised glass microscope slides, onto which a 50 μl aliquot of spores (containing 10⁴-10⁸ spores ml⁻¹) was added to the antibody-coated region of the slide and permitted to bind for 1 hour. Binding time has subsequently been reduced to 10 minutes with no apparent reduction in binding efficacy. Following washing and confirmation of successful binding of spores to the antibody-labelled surface, germination was induced by addition of 50 μl Tris buffer (50-mM), containing 1-mM L-alanine. Micrographs showed the transition from phase bright spores to phase dark that characterises the germination response, indicating that immobilisation does not impede germination. Subsequent studies with spore suspensions demonstrated that spore density did not appear to influence germination kinetics, meaning that even low numbers of captured spores should germinate at a similar rate to those captured at high density. Considered together, these studies suggest that spores immobilised by antibodies in the vicinity of the holographic matrix should display normal germination kinetics; this is regardless of spore density, when induced to germinate upon addition of germinant, a crucial parameter for the final instrument design.

In a preferred system according to the invention, that exploits the activation of CwlJ in situ, i.e. by incorporating intact spores into the sensor design, Ca-DPA released from germinating target spores (captured by specific antibodies) stimulates CwlJ-mediated germination of sensor-associated spores. Ca-DPA released from these spores, which are essentially transducing and amplifying the target spore germination response, can then be detected by a RNTA (or similar) holographic sensor. The spores themselves may either be immobilised in the vicinity of the sensor or embedded in the polymer itself. Furthermore, since germination can only be triggered by Ca-DPA (which is unique in nature to bacterial spores), and not free Ca²⁺ ions, a further element of specificity is introduced to the system. The methodology for synthesis of this ligand and its application for the detection of spores is described in Bhatta et al, Biosensors and Bioelectronics 23 (2007) 520-527, the content of which is incorporated herein by reference.

The amplification spores have to meet a number of criteria before being considered for inclusion in such a sensor system. In particular, the signal amplification spores must not respond to the same nutrient germinants applied to germinate the target spores. Bacillus anthracis, for example, germinates most efficiently when exposed to a mixture of L-alanine and inosine (alanine is the major germinant for most Bacillus species). This problem can be circumvented by engineering “receptorless” spores that can only germinate in response to exogenous Ca-DPA. The signal amplification spores should also be sufficiently sensitive to relatively low concentrations of exogenous Ca-DPA that the signal amplification cascade is triggered by low numbers of germinating target spores.

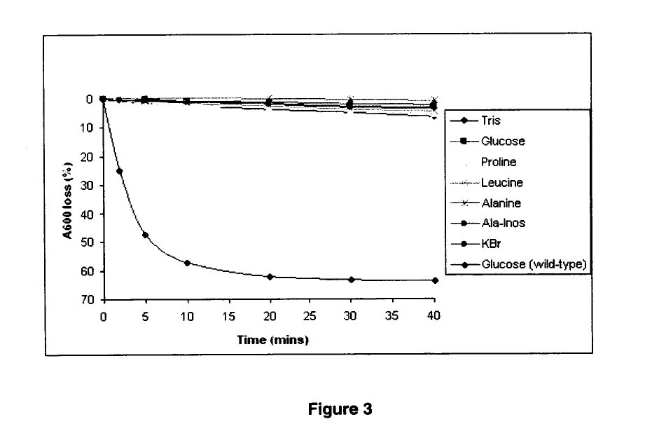

It has been found that spores of Bacillus megaterium QM B1551 germinate very synchronously and rapidly compared to other routinely employed laboratory strains, e.g. Bacillus subtilis and Bacillus megaterium KM. Spores of Bacillus megaterium QM B1551 germinate in response to a number of compounds, including glucose, various amino acids, and a number of salts (so called “ionic” germination). A strain has been constructed that carries a mutation in one of the GerA type operons that encodes the putative germinant receptors. Spores of this strain have lost the ability to respond to all germinant triggers tested to date (including alanine), as illustrated in FIG. 3.

Construction of these spores (strain GC417) is described in Christie et al, J. Bacteriology, vol. 189, no. 12 (June 2007) 4375-4383, the content of which is incorporated herein by reference. These spores may germinate slowly in response to a complex mixture of nutrients but do not respond to single trigger compounds, and such spores are suitable for use in the invention.

These data demonstrate that receptorless mutants fail to germinate upon exposure to a range of nutrient germinants. The wild-type response to glucose is included for comparative purposes. The slight decrease observed in the A600 of the mutant spores is due to spore clumping rather than any degree of germination.

The CwlJ gene has been sequenced (GenBank accession number EU037904). Unlike Bacillus subtilis, the CwlJ gene appears to be organised in a bi-cistronic operon with a homologue of GerQ.

GerQ is required for the localisation of CwlJ in the spore coat layer. The fact that CwlJ and GerQ are organised as a single transcriptional unit in Bacillus megaterium makes it simple to over-express both genes, to increase the sensitivity of the spores to Ca-DPA. Studies conducted to date have demonstrated that both receptorless and wild-type Bacillus megaterium OM B1551 spores have an apparent Km (the concentration of Ca-DPA that induces a half-maximal germination response) of approximately 35 mM. This value is similar to that previously observed in Bacillus subtilis. Further, a CwlJ knock-out strain of Bacillus megaterium does not germinate in response to exogenous Ca-DPA, demonstrating that Ca-DPA induced germination is mediated via this enzyme in this species.

In addition, a multi-copy plasmid carrying a copy of the Bacillus megaterium CwlJ/GerQ operon (including upstream regulatory sequences) has been constructed and introduced into wild-type and receptorless cells. Spores carrying the plasmid may lead to over-expression of CwlJ, are currently being prepared and will be tested for sensitivity to exogenous Ca-DPA. A second construct carries a copy of the CwlJ operon under the control of a high copy number promoter that is recognised by the appropriate RNA polymerase (E sigma E). When this construct is integrated into the chromosome, spores can be prepared in the absence of antibiotic (which are generally required to maintain the presence of freely replicating plasmids), which is the preferred method.

The following Example illustrates the invention.

### EXAMPLE

SASPs extracted from mature B. megaterium spores by acid-rupture (Johnson and Tipper, 1981) were cross-linked with formaldehyde to form stable hydrogel films coated on silanised glass slides. Films were then immersed sequentially in solutions of silver nitrate and lithium bromide containing a photosensitising dye (Blyth et al., 1999). Following exposure to laser light and conventional photographic development, an interference fringe pattern comprising layers of ultra-fine (<20 nm diameter) grains of metallic silver distributed within the thickness of the polymer film is generated, thereby transforming polymer films into reflection holograms. Illumination of the developed grating under white light results in a characteristic spectral peak whose wavelength is governed by the Bragg equation. Following extensive hydration with deionised water to induce polymer swelling, SASP-based holograms were visually perceptible, reflecting predominantly bright green light.

More specifically, miniature SASP polymer films (6×10 mm) were formed on aminosilanised glass slides from 10 μl aliquots of a solution comprising 12% (w/v) SASPs in 1% acetic acid, which were cross-linked in a chamber enriched with formaldehyde vapours for 3 min. Films were transformed into holograms using the diffusion method outlined in Blyth et al., (1999), i.e. sequential exposure to 0.3 M silver nitrate solution and 3% (w/v) lithium bromide solution containing 0.1% (w/v) OBS (1,1 -diethyl-2,2-cyanine iodide). Photosensitised films were exposed to three 10 ns pulses from a frequency doubled Nd/YAG laser (350 mJ, 532 nm, Brilliant B, Quantel, France), immersed in Saxby developer (Saxby, 1994) and fixed in 10% (w/v) sodium thiosulphate. Holograms were rinsed in water to aid visualisation by eye under spotlight illumination.

Holograms were interrogated using an in-house reflection spectrometer as described in Mayes et al. (1998). Degradation of SASP-based holographic matrices was investigated using GPR^{S}, a variant of B. megaterium GPR that undergoes faster Ca-DPA²⁺-stimulated processing from its inactive zymogen to the catalytically active enzyme (Illades-Aguiar and Setlow, 1994b). Expression and purification of GPR^{S} from E. coli PSI 909 cells carrying the B. megaterium gpr^{s} gene (Illades-Aguiar and Setlow, 1994b) was modified from Sanchez-Salas and Setlow (1993). Recombinant GPR^{S}, purified to 95% homogeneity by ion-exchange chromatography, was processed to its catalytically active form by incubation with Ca²⁺-DPA as described by Illades-Aguiar and Setlow (1994a). The effect of Ca²⁺-DPA-activated GPR^{S} on the diffraction characteristics of an SASP-based hologram was monitored: following an initial >10-minute delay, a steady deterioration in peak intensity was observed over a time period of 1.5-2 hours; stabilisation of the holographic profile occurred when the peak was between 10-50% of its starting intensity. The original spectral peak could not be restored despite extensive washing with equilibration buffer, demonstrating the irreversibility of the holographic response; this is attributed to enzymatic matrix degradation compromising the integrity of the holographic fringes. The reduction in peak intensity is due to disordering and progressive loss of holographic fringes to the solution as the SASP-based matrix is degraded by GPR^{S}. The effect of inactive GPR^{S} (i.e. pre-incubation with Ca²⁺-DPA) on holographic diffraction characteristics was also investigated and revealed no significant changes in the holographic signal over time. This observation is attributed to failure of the enzyme to degrade the holographic matrix and is consistent with the lack of catalytic activity of GPR prior to Ca²⁺-DPA-stimulated auto-processing (Illades-Aguiar and Setlow, 1994a). The same holographic matrix was subsequently degraded by activated GPR^{S}, demonstrating the inherent responsiveness of the sensor.

### REFERENCES

Blyth et al. 1999. The Imaging Science Journal 47, 87-91.
Illades-Aguiar and Setlow, 1994a. Journal of Bacteriology 176, 7032-7037.
Illades-Aguiar and Setlow, 1994b. Journal of Bacteriology 176, 2788-2795.
Johnson and Tipper, 1981. Journal of Bacteriology 146, 972-982.
Mayes et al. 1998. Journal of Molecular Recognition 11, 168-174.
Sanchez-Salas and Setlow, 1993. Journal of Bacteriology 175, 2568-2577.
Saxby, 1994. Practical Holography, Prentice Hall, Englewood Cliffs.

1. A sensor for spores, comprising spore-binding ligands and, on or within the body of the sensor, a material that is responsive to Ca-DPA (calcium-dipicolinic acid) but not to a (or another) germinant.

2. The sensor according to claim 1, wherein the ligand is an immobilized antibody.

3. The sensor according to claim 1, wherein the material comprises spores.

4. The sensor according to claim 1, which comprises a matrix and a hologram recorded therein, wherein a change in a property of the matrix caused directly or indirectly by the response of said material to Ca-DPA can be observed.

5. A method for the detection of spores in a sample, comprising
contacting the sample with a sensor according to claim 1, whereby spores in the sample are bound by the ligands;
introducing the germinant; and
observing the response of the material.
